(19) United States
(12) Patent Application Publication
(10) Pub. No.: US 2016/0206247 A1
(43) Pub. Date: Jul. 21, 2016

(54) ADAPTIVE MOTION CORRECTION IN PHOTOPLETHYSMOGRAPHY USING REFERENCE SIGNALS

(71) Applicant: Covidien LP, Mansfield, MA (US)

(72) Inventors: Eric Morland, Erie, CO (US); Clark R. Baker, JR., Newman, CA (US); Daniel Lisogurski, Boulder, CO (US); Rasoul Yousefi, Superior, CO (US); Philip Davis, El Paso, TX (US); Christopher J. Meehan, Arvada, CO (US)

(21) Appl. No.: 14/996,471

(22) Filed: Jan. 15, 2016

Related U.S. Application Data
(60) Provisional application No. 62/105,917, filed on Jan. 21, 2015.

(51) Int. Cl. A61B 5/00; A61B 5/024

(52) U.S. Cl. CPC A61B 5/7207; A61B 5/7225; A61B 5/02427

(57) ABSTRACT

Systems, methods, apparatuses, and software for providing enhanced measurement and correction of physiological data are provided herein. In a first example, a physiological measurement system is configured to obtain a measured photoplethysmogram (PPG) for a patient, and obtain a reference signal for the patient measured concurrent with the measured PPG, the reference signal including noise components related to at least motion of the patient. The physiological measurement system also is configured to determine a filtered PPG from the measured PPG using at least an adaptive filter with the reference signal to reduce noise components of the measured PPG, determine a final PPG by spectrally subtracting at least a portion of the noise components of the reference signal from the filtered PPG, and identify one or more physiological metrics of the patient based on the final PPG.

### RELATED APPLICATIONS

This application hereby claims the benefit of priority to U.S. Provisional Patent Application 62/105,917, titled “ADAPTIVE MOTION CORRECTION IN PHOTOPLETHYSMOGRAPHY USING REFERENCE SIGNALS,” filed Jan. 21, 2015, which is hereby incorporated by reference in its entirety.

### TECHNICAL FIELD

Aspects of the disclosure are related to the field of medical devices, and in particular, measuring physiological parameters and correcting measured physiological parameters, such as plethysmograms.

### BACKGROUND

Various medical devices can non-invasively measure parameters of blood in a patient. Pulse oximetry devices are one such non-invasive measurement device, typically employing solid-state lighting elements, such as light-emitting diodes (LEDs) or lasers, to introduce light into the tissue of a patient. The light is then detected to generate a photoplethysmogram (PPG). These photoplethysmography systems can also measure changes in blood volume of tissue of a patient and calculate various parameters such as heart rate, respiration rate, and oxygen saturation.

However, conventional optical pulse oximetry devices are subject to motion noise and other inconsistencies which limits the accuracy of such devices. For example, motion of the patient and movement of nearby objects or medical personnel can lead to signal corruption and inaccuracies in optical-based measurements. These inaccuracies in the optical-based measurements can then lead to inaccurate oxygen saturation level calculations, false pulse reporting, or prevent measurement of the patient until motion noise subsides. Motion of the patient can be especially troublesome in neonatal intensive care units (NICU) due to the often uncontrollable movements of infants in incubators or other medical environments.

Capacitive sensing has been employed to measure some physiological parameters by applying electric fields to the tissue of the patient. However, these capacitive systems can still suffer from noise and inconsistencies due to not only motion of the patient, but also motion of nearby objects and personnel.

### OVERVIEW

Systems, methods, apparatuses, and software for providing enhanced measurement and correction of physiological data are provided herein. In a first example, a physiological measurement system is provided. The physiological measurement system includes an optical system configured to obtain a measured PPG for a patient, and a reference system configured to obtain a reference signal for the patient measured concurrent with the measured PPG, where the reference signal includes noise components related to at least motion of the patient. The physiological measurement system also includes a signal processing system configured to determine a filtered PPG from the measured PPG using at least an adaptive filter with the reference signal to reduce noise components of the measured PPG. The signal processing system is configured to determine a final PPG by spectrally subtracting at least a portion of the noise components of the reference signal from the filtered PPG, and identify one or more physiological metrics of the patient based on the final PPG.

In a second example, a method of operating a physiological measurement system is provided. The method includes obtaining a measured PPG for a patient, and obtaining a reference signal for the patient measured concurrent with the measured PPG, the reference signal including noise components related to at least motion of the patient. The method also includes determining a filtered PPG from the measured PPG using at least an adaptive filter with the reference signal to reduce noise components of the measured PPG, determining a final PPG by spectrally subtracting at least a portion of the noise components of the reference signal from the filtered PPG, and identifying one or more physiological metrics of the patient based on the final PPG.

In a third example, an apparatus is provided comprising one or more computer readable storage media and program instructions stored on the one or more computer readable storage media for at least identifying one or more physiological metrics of a patient. When executed by a processing system the program instructions direct the processing system to obtain a measured PPG for a patient, and obtain a capacitance signal for the patient measured concurrent with the measured PPG using one or more capacitive plates proximate to tissue of the patient, the capacitance signal including noise components related to at least motion of the patient. The program instructions also direct the processing system to determine a filtered PPG from the measured PPG using at least an adaptive filter with the capacitance signal to reduce noise components of the measured PPG, determine a final PPG by spectrally subtracting at least a portion of the noise components of the capacitance signal from the filtered PPG, and identify the one or more physiological metrics of the patient based on the final PPG.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 is a system diagram illustrating a physiological measurement system.

FIG. 2 is a flow diagram illustrating a method of operating a physiological measurement system.

FIG. 3 is a system diagram illustrating a physiological measurement system.

FIG. 4 illustrates various measured signals for a patient.

FIG. 5 is a flow diagram illustrating a method of operating a physiological measurement system.

FIG. 6 illustrates various measured signals for a patient.

FIG. 7 is a system diagram illustrating physiological measurement systems.

FIG. 8 is a system diagram illustrating a physiological measurement system.

FIG. 9 illustrates various measured signals for a patient.

FIG. 10 is a block diagram illustrating a physiological measurement system.

### DETAILED DESCRIPTION

The examples discussed herein include systems, apparatuses, methods, and software for enhanced measurement of physiological parameters in patients. When certain measurements of patient data are performed, such as optical measurements, signals associated with the measurements can be subjected to various interference and noise due to patient motion, among other sources of noise. For example, motion noise occurs in pulse oximetry measurements due in part to optical emitter-detector spacing changes, light coupling changes, deformation of the tissue under measurement, and changes in venous blood volume, among other motion noise sources. It can be difficult to reduce the noise caused by motion during optical measurements. However, capacitance-based sensing or ambient light sensing can be employed as reference signals in conjunction with optical measurements of a photoplethysmogram (PPG) to provide for effective filtering and noise correction of the PPG. These reference signals can be employed with adaptive filtering and signal processing to provide motion correction, noise filtering, data stabilization, or add additional sensing capabilities to measurement systems.

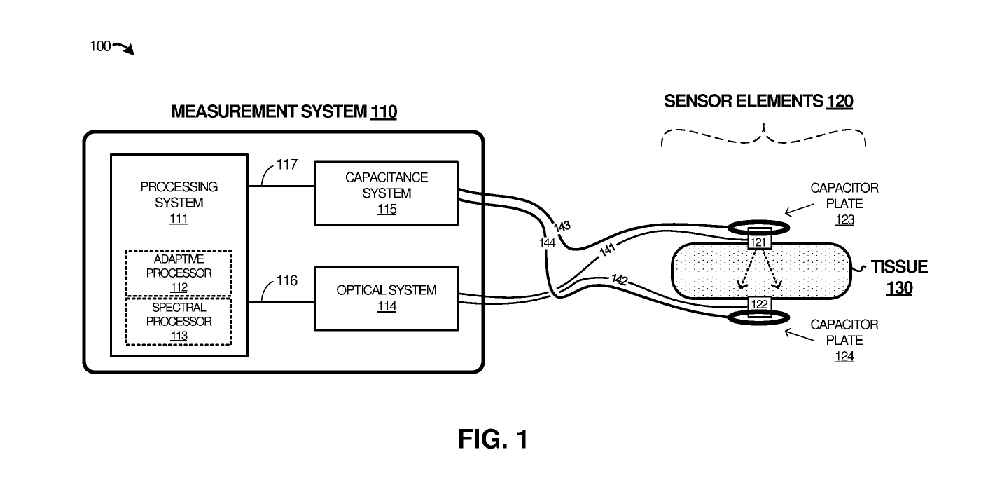

As a first example of a measurement system for monitoring physiological parameters of a patient, FIG. 1 is presented. FIG. 1 is a system diagram illustrating physiological system 100. Elements of physiological system 100 measure one or more physiological parameters of tissue 130, and can comprise measurement equipment, apparatuses, and systems which can be used in a patient care setting. Some of the elements of FIG. 1 may be included in implantable medical devices. Physiological system 100 includes measurement system 110, sensor elements 120, and tissue 130. In operation, sensor elements 120 are configured to monitor various properties of tissue 130 and provide signals indicating these properties to measurement system 110 for processing and analysis.

Measurement system 110 includes processing system 111 which further includes adaptive processor 112 and spectral processor 113. Measurement system 110 also includes optical system 114 and capacitance system 115. Optical system 114 and processing system 111 communicate over link 116. Capacitance system 115 and processing system 111 communicate over link 117. Links 116 and 117 can each comprise one or more analog or digital links.

Optical system 114 measures various properties of tissue 130 using optical emitter 121 and optical detector 122. In some examples, optical system 114, along with optical emitter 121 and optical detector 122, comprise a pulse oximeter and can measure a PPG for tissue 130. This PPG can be used to determine various properties of tissue 130 or the patient associated with tissue 130, such as changes in blood volume of tissue 130 which correspond to various parameters such as pulse rate, respiration rate, and oxygen saturation, among other parameters.

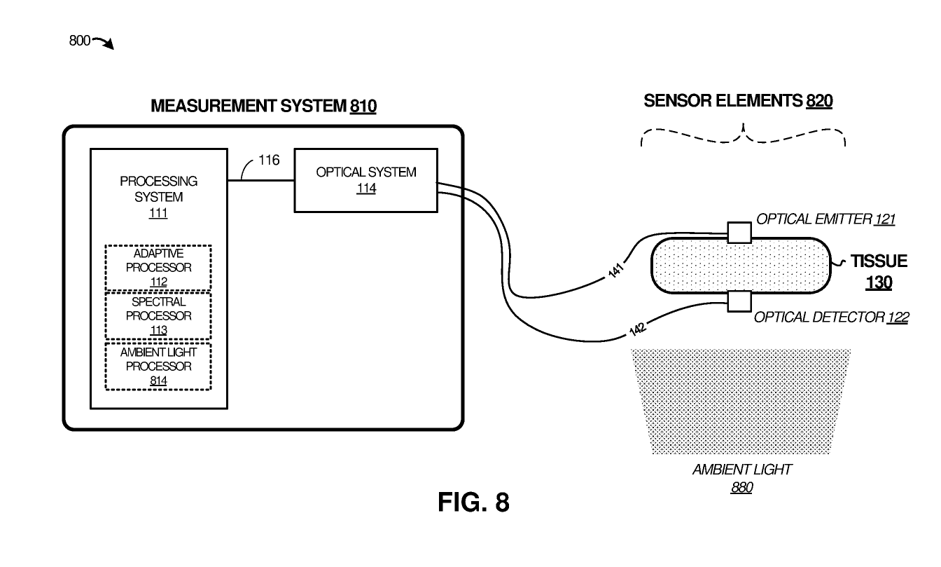

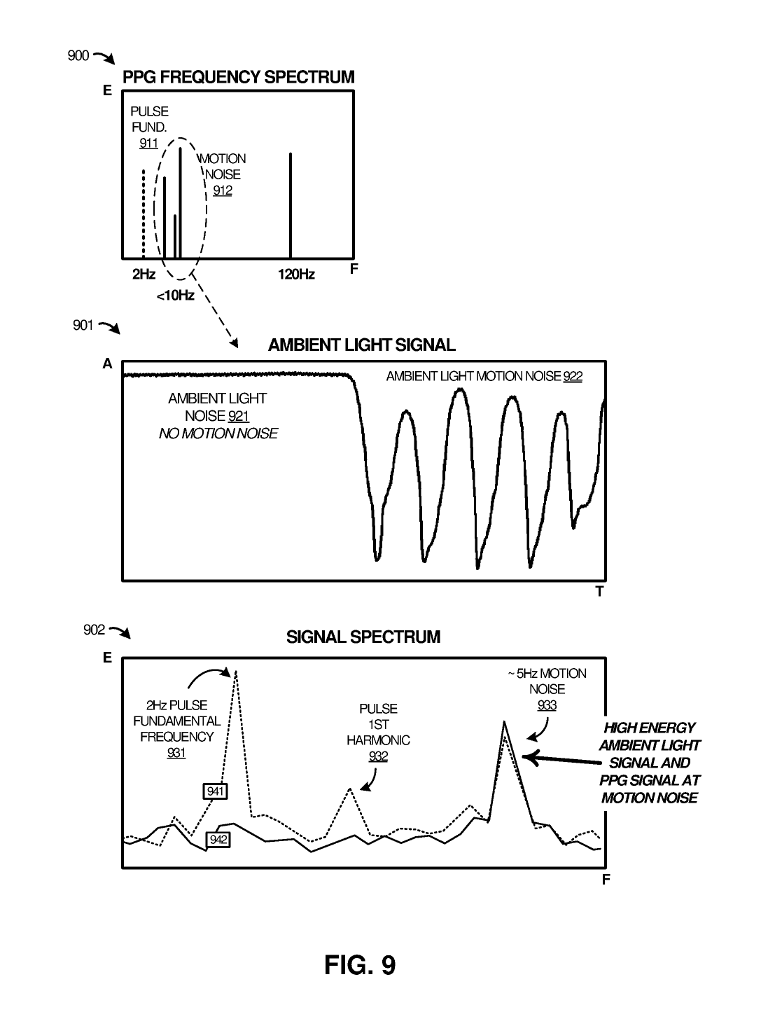

However, motion of tissue 130 as well as other bulk motion of the patient can introduce noise and signal corruption into measurements done by optical system 114. A reference system is employed in FIG. 1 to provide at least one secondary signal which can be used to reduce noise in the optical signals. In FIG. 1, the reference system is capacitance system 115. Other examples of reference systems are possible, such as that discussed in FIGS. 8-9 below which uses ambient light signals instead of capacitance signals.

Turning now to capacitance system 115, capacitance signals associated with capacitor plates 123-124 is monitored concurrent with optical measurement of tissue 130. Capacitance system 115 monitors changes in capacitance for capacitor plates 123-124 which can indicate motion of tissue 130 among other signals representative of physiological properties. To measure the changes in capacitance, capacitance system 115 can drive an oscillating or alternating current (AC) signal onto link 143 for emission by capacitor plate 123 proximate to tissue 130. Likewise, capacitance system 115 can drive an oscillating AC signal onto link 144 for emission by capacitor plate 124 proximate to tissue 130. Changes in signals driven onto link 143-144, such as current draws, can correspond to capacitance value changes. Differential and single-ended measurement schemes may be employed, and one of capacitor plates 123-124 can be electrically grounded. A single capacitive element might be employed instead of the dual capacitive configuration shown in FIG. 1. Furthermore, a single-sided capacitance measurement using one or two capacitor plates on the same side of tissue 130 can be employed. The generally circular ring shapes of capacitor plates 123-124 might vary and instead be rectangular-shaped rings, triangular rings, or oval or elliptical rings, among other shapes and sizes. Non-ring shaped capacitor plates can instead be employed.

Returning to the optical measurement elements of FIG. 1, optical system 114 drives signals over link 141 to optical emitter 121. Optical emitter 121 emits optical signals into tissue 130 for propagation through tissue 130. Optical detector 122 detects these optical signals after propagation through tissue 130. Optical system 114 receives signals over link 142 from optical detector 122. The signals on links 141 and 142 can comprise optical signals when links 141 and 142 comprise optical fiber links, or can comprise electrical signals when links 141 and 142 comprise electrical links. Various combinations of optical and electrical signaling can be employed between any of optical emitter 121 and optical detector 122 and optical system 114.

In some examples, link 141 is a wired or wireless signal link, and carries a measurement signal to optical emitter 121, and optical emitter 121 converts the measurement signal into an optical signal and emits an optical signal into tissue 130. The optical signal can be emitted using a laser, laser diode, light emitting diode (LED), or other light emission device. In other examples, link 141 is an optical link, and carries an optical signal to optical emitter 121. Optical emitter 121 can comprise tissue interface optics, such as lenses, prisms, or other optical fiber-to-tissue optics, which interface to tissue 130 for emission of optical signals. One or more optical wavelengths can be introduced by optical emitter 121 into tissue 130, and the one or more optical wavelengths can be selected based on various physiological factors, such as isosbestic wavelengths associated with blood components of tissue 130. In a particular example, wavelengths such as 660 nm and 808 nm are employed.

Optical detector 122 detects the optical signals after propagation through tissue 130. Optical system 114 receives signals over link 142 from optical detector 122 representative of the optical signal after propagation through tissue 130. In some examples, link 142 is a wired or wireless signal link, and carries a signal from optical detector 122, where optical detector 122 converts detected optical signals into associated electrical signals. Detector 122 can comprise a photodiode, avalanche photodiode, or other optical detection device. In other examples, link 142 is an optical link, and carries an optical signal from optical detector 122. Optical detector 122 can comprise tissue interface optics, such as described above for optical emitter 121, which interface to tissue 130.

As mentioned above, processing system 111 includes adaptive processor 112 and spectral processor 113. These elements can comprise discrete electronics and discrete logic devices, or can comprise software elements, including combinations thereof. Adaptive processor 112 includes one or more adaptive filtering elements which can process capacitance signals monitored by capacitance system 115 and optical signals monitored by optical system 114 to reduce noise levels in the optical signals. Spectral processor 113 includes one or more signal processing elements which can perform additive or subtractive signal operations in a time domain or a frequency domain. Further signal processing elements can be included in processing system 111, such as digital signal processors (DSP), finite impulse response (FIR) filters, amplifiers, low pass filters, high pass filters, or bandpass filters, among other signal handling and processing elements.

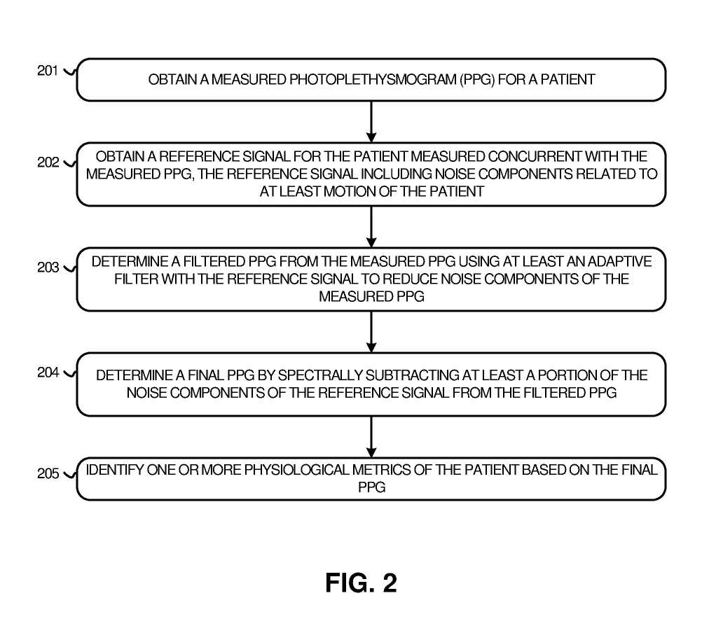

To illustrate the operation of the elements of FIG. 1, FIG. 2 is provided. FIG. 2 is a flow diagram illustrating a method of operating measurement system 110. The operations of FIG. 2 are referenced below parenthetically.

In FIG. 2, optical system 114 obtains (201) a measured PPG for a patient. The PPG comprises data which is measured by optical system 114 for tissue 130. In this example, optical system 114 actually measures tissue 130 using optical emitter 121 and optical detector 122. In other examples, this PPG can be measured by other systems external to measurement system 110 and measurement system 110 can receive the PPG or data comprising the PPG from these other systems.

To measure the PPG, optical emitter 121 emits light into tissue 130 which is then detected by optical detector 122. Optical emitter 121 receives signals over link 141 which direct optical emitter 121 to emit light into tissue 130. This light can comprise one or more modulated optical signals, composed of one or more wavelengths. Signals representative of the light detected by optical detector 122 are transferred over link 142 for processing by optical system 114 and subsequent identification of the PPG.

The measured PPG comprises a pulsatile signal representative of a pulse of the patient, as measured through tissue 130. In addition to the pulsatile signals, other signals, such as noise can be measured by optical system 114. This noise can be caused by motion of the patient, such as by tapping, squeezing, or normal movements of the patient during optical measurement. However, in addition to the measured PPG for the patient, a reference signal is also measured. In this example, the reference signal is a capacitance signal, although other reference signals can be employed.

Capacitance system 115 obtains (202) the reference signal for the patient measured concurrent with the measured PPG, the reference signal including noise components related to at least motion of the patient. Although the capacitance signal is measured by capacitance system 115, other examples can instead employ external capacitance measurement systems, and processing system 111 can receive data representative of the capacitance signal from the external capacitance measurement systems.

To measure the capacitance signal, many different methods of capacitance-based measurement might be employed. In FIG. 1, two ring-shaped capacitor plates are employed and each are driven with a separate modulated signal. These signals are monitored by capacitance system 115 to identify capacitance value changes over time which are then processed into a differential capacitance signal. In other examples, one of capacitor plates 123-124 is coupled to a reference potential, such as a ground potential, and the other one of the capacitor plates is driven with an electrical signal. Capacitance changes for the driven one of the capacitor plates is monitored to identify the capacitance signal. Other techniques for driving capacitor plates 123-124 and measuring capacitance value changes for capacitor plates 123-124 can be employed, including omitting one of capacitor plates 123-124 or placing both of capacitor plates 123-124 on the same side of tissue 130.

As mentioned above, the examples discussed herein employ one or more generally ring-shaped capacitor plates. These ring-shaped capacitor plates maximize sensitivity to motion noise, but minimize sensitivity to changes in venous blood volume of tissue 130, such as due to pulse. Signals measured by the ring-shaped capacitor plates include motion noise which can be used to cancel out or reduce similar motion noise in concurrent optical signals measured to obtain the PPG. The ring-shaped capacitor plates allow pulsatile changes in the tissue under measurement to minimally affect the capacitor plates, while still allowing for detection of bulk movements of the tissue, such as due to pressing, squeezing, flexing, and clenching of tissue 130. Also, an asymmetric gain can be applied to the ring-shaped capacitor plates, which can enhance signal detection when motion noise occurs equally on two ring-shaped capacitor plates which might be otherwise be canceled out in a differential detection mode.

Processing system 110 determines (203) a filtered PPG from the measured PPG using at least an adaptive filter with the reference signal to reduce noise components of the measured PPG. The filtered PPG will have a reduced level of motion noise compared to the measured PPG, and thus be a cleaner PPG which can be used in further processing, such as operation 204, or to identify one or more physiological metrics, such as in operation 205. As mentioned above, the reference signal is the capacitance signal measured for tissue 130 in FIG. 1.

The adaptive filter can include adaptive processor 112 of processing system 111, although other adaptive filter elements can be employed. Adaptive processor 112 can include noise cancellation filters, such as finite impulse response (FIR) filters, recursive least squares (RLS) filters, least means squares (LMS) filters, or normalized least mean squares (NMLS) filters, among others, including combinations thereof. Parameters for the adaptive filtering portions of adaptive processor 112 can be modified based on feedback of measured or past measured physiological metrics of the patient, such as a pulse rate, breathing rate, or other parameters. These parameters can be used to adjust filtering properties over time in concert to ensure various fundamental frequencies associated with the measured PPG are targeted properly by the adaptive filtering elements.

In the example of a FIR filter, the reference signal is employed in a noise cancellation process for the measured PPG, where noise components of the reference signal can cancel out noise components of the PPG to identify the filtered PPG. The FIR filter is configured to process the reference signal against an error signal based on the filtered PPG to identify a cancellation signal, and sum the cancellation signal with the measured PPG to identify the filtered PPG.

In an example of a RLS filter, the RLS filter recursively finds filter coefficients that minimized a weighted linear least squares cost function relating to input signals. The input signals are the measured PPG having motion noise and the capacitance signal which is representative of the motion noise. RLS filters can provide fast convergence, although RLS filters have larger computational loads than other filters.

In other examples of adaptive processor 112, blind source separation techniques are employed using two input signals, namely the measured PPG and the capacitance signal. The blind source separation can include processing the measured PPG with a statistical independent component analysis (ICA) to extract statistically independent signals from each of the measured PPG and capacitance signal. The extracted statistically independent signals are a first signal representing motion noise of the patient and a second signal comprising the filtered PPG.

Once the filtered PPG is determined, processing system 110 determines (204) a final PPG by spectrally subtracting at least a portion of the noise components of the reference signal from the filtered PPG. Although the filtered PPG is a cleaner waveform as compared to the measured PPG, further processing is performed on the filtered PPG. This further processing of operation 204 is performed in the frequency domain. Noise components of the capacitance signal are identified and subtracted from correlated components of the filtered PPG.

The noise components can be identified as having higher energy in the capacitance signal than pulsatile components, or having frequencies that do not correspond to pulsatile components. For example, the pulsatile components can be identified as having one or more fundamental frequencies with associated harmonics. These fundamental frequencies may change over time, but over a particular timeframe the fundamental frequencies can be used to identify which signal components of the capacitance signal are most likely associated with motion noise and which are most likely not associated with motion noise. Spectral content of the capacitance signal that is associated with motion noise is subtracted in the frequency domain from the filtered PPG. Various scaling or gain factors can be applied to the spectral content of the capacitance signal that is associated with motion noise. In some examples, a scaled version of the entire capacitance signal is spectrally subtracted from the filtered PPG.

Responsive to determining the final PPG, processing system 110 identifies (205) one or more physiological metrics of the patient based on the final PPG. The physiological metrics can include various plethysmograph (pleth) information, such as clean photoplethysmograms (PPG) and temporal variability of PPG parameters (such as pleth morphology and pulse information). The physiological metrics can also include electrocardiography (ECG) information via capacitive sensing, pulse rate, respiratory rate, respiratory effort, blood pressure, oxygen concentrations, hemoglobin concentrations, total hemoglobin concentration (tHb), saturation of peripheral oxygen (SpO₂), SpO₂ variability, regional oxygen saturation (rSO₂), apnea conditions, arrhythmia, non-invasive hematocrit, carboxyhemoglobin concentration, methemoglobin concentration, and saturation pattern detection among other parameters and characteristics, including combinations and variations thereof.

Physiological measurements can be performed using the final PPG. Some of these include determining respiration rate from a finger, pulse rate from a finger, motion of patient, continuous non-invasive blood pressure measurement (CNIBP), cardiac output (CO), pulse transit time (PPT) deltaPOP (a measurement of the variability of the pleth pulses), variability of optical pleth to determine vessel elasticity, dehydration, apnea detection and monitoring, and auto-regulation of patients. Physiological metric identification and physiological measurements can be performed using both optical and capacitance-based signals as well. A “PMOD,” or percentage modulation, of the various signals monitored for the patient can also be determined. PMOD comprises oscillation amplitude relative to the baseline amplitude for a particular signal.

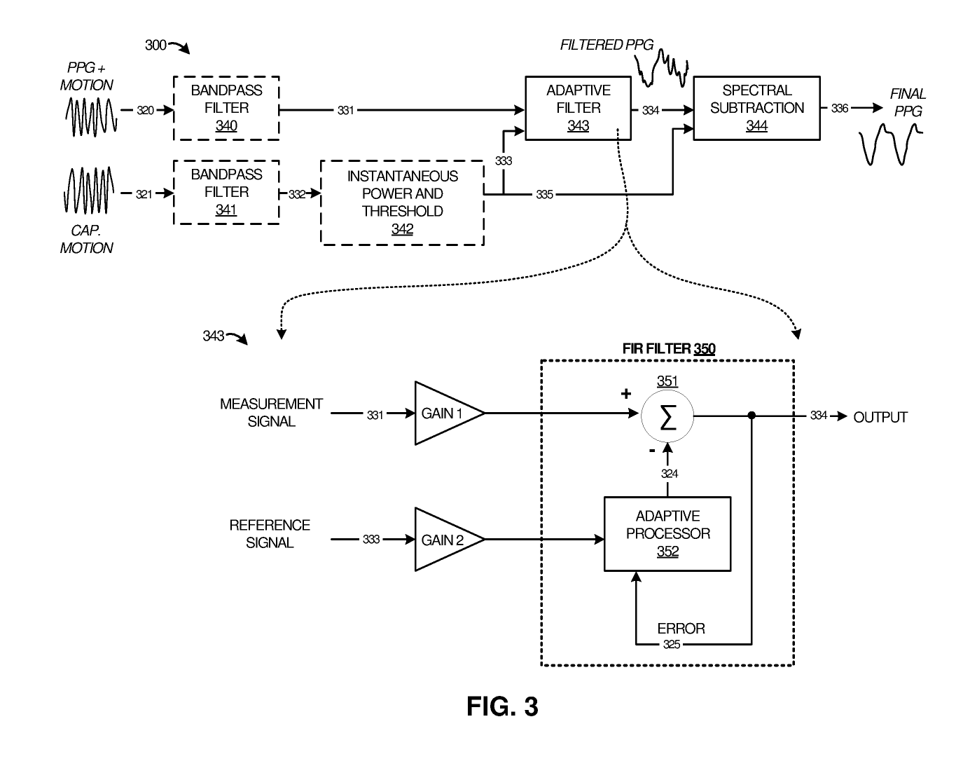

To provide further examples of adaptive filtering and spectral subtraction discussed in FIGS. 1 and 2, signal processor 300 is presented in FIG. 3. Signal processor 300 includes several modular elements, namely bandpass filter 340, bandpass filter 341, instantaneous power and threshold element 342, adaptive filter 343, and spectral subtraction element 344. Each of the elements of signal processor 300 can comprise hardware signal processing elements, such as filters and amplifiers, or can comprise software signal processing elements, including combinations thereof. Bandpass filters 340-341 and instantaneous power and threshold element 342 can be omitted in some examples.

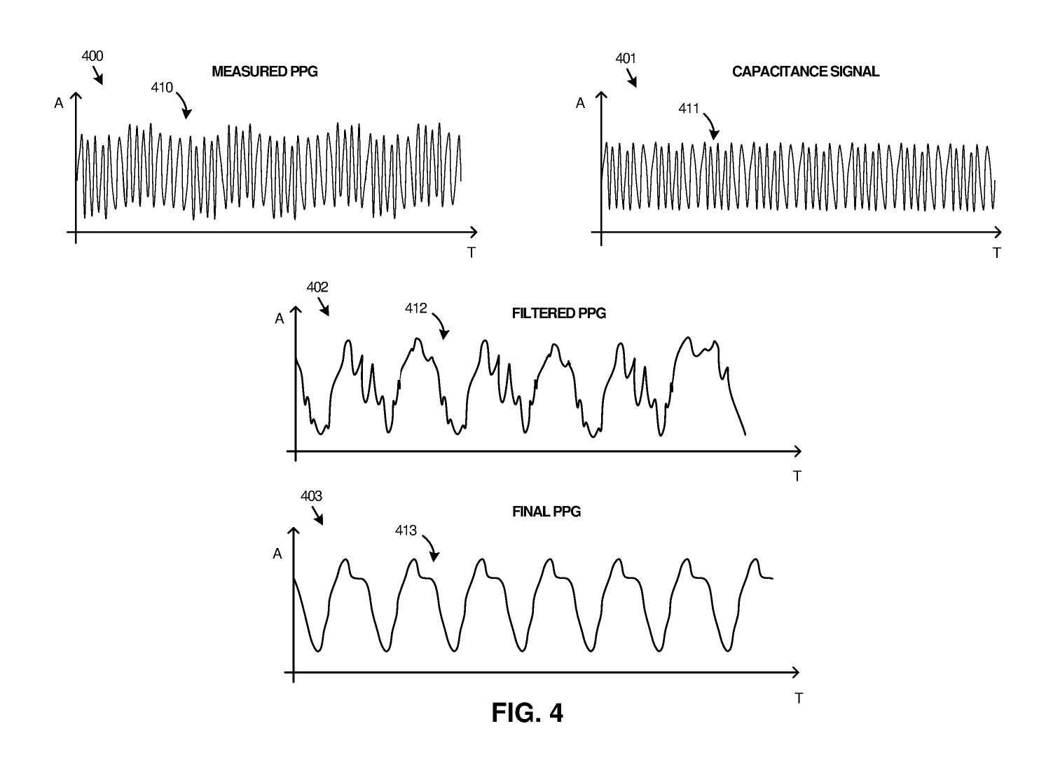

In FIG. 3, signal 320 representative of a PPG that includes motion noise is introduced to bandpass filter 340. Signal 320 can be an optically measured signal for tissue of a patient, such as that measured by optical system 114 of FIG. 1. FIG. 4 shows one example of signal 320 in graph 400, namely measured PPG 410. As can be seen in FIG. 4, measured PPG 410 includes noise attributed to motion of the patient during measurement. Bandpass filter 340 can remove some noise components of signal 320 prior to further processing, such as to remove low and high frequency content which is outside of a predetermined frequency range of the pulsatile signals that are being measured.

Similarly, signal 321 representative of motion of the patient is introduced into bandpass filter 341. A similar bandpass range can be employed for signal 321 to remove frequencies outside of a range of interest, such as DC content as well as high-frequency AC content. Signal 321 can be a capacitance signal as measured by a capacitor system, such as capacitor system 115 of FIG. 1. Signal 321 can instead be an ambient light signal as discussed in FIGS. 8-9. FIG. 4 shows one example of signal 321 in graph 401, namely capacitance signal 411. As can be seen in FIG. 4, capacitance signal 411 also includes noise attributed to motion of the patient during measurement.

Filtered signal 331 is provided to adaptive filter 343 along with signal 333. Signal 333 is optionally passed through element 342. Element 342 can provide for further filtering of the capacitance signal to eliminate or ignore content of signal 332 which fails to pass power thresholds. For example, when motion noise indicated by signal 332 is below a power threshold, further processing by elements 343 and 344 might not be desired since a relatively clean PPG has already been obtained in signal 320. This can arise when motion of the patient is not occurring, or is occurring below a predetermined threshold. In further examples, an instantaneous power threshold can be applied to signal 332, which can be obtained by applying a Hilbert filter to signal 332 and computing the absolute value of the resulting signal. Signal 333 can be set to zero when the instantaneous power of signal 332 fails to pass the instantaneous power threshold. This can reduce the risk of altering a relatively clean PPG signal when there is little noise from motion in signal 332.

Adaptive filter 343 can include various noise cancellation filters, such as FIR filters, RLS filters, LMS filters, or NMLS filters, among others, including combinations thereof. In the example in FIG. 3, FIR filter 350 is shown. FIR filter 350 includes summation element 351 and adaptive processor 352. Signals 331 and 333 are provided into optional gain elements which can increase or decrease a signal level of signals 331 and 333. Signal 331 is employed as a measurement signal in FIG. 3, while signal 333 is employed as a reference signal in FIG. 3.

In FIR filter 350, the reference signal is employed in a noise cancellation process for the measurement signal, where noise components of the reference signal can cancel out noise components of the measurement signal to identify a filtered signal, namely a filtered PPG in this example. Adaptive processor 352 is configured to process the reference signal against error signal 325 to identify cancellation signal 324, and sum cancellation signal 324 with the measurement signal to identify output signal 334. In this example, error signal 325 comprises a feedback of output signal 334. In FIG. 3, output signal 334 comprises a filtered PPG, and an example of this signal is included in graph 402 of FIG. 4, namely filtered PPG 412.

In further examples, of signal processor 300, a bank of adaptive filters can be included, with each of the adaptive filters corresponding to a different frequency band. Each frequency band can have an associated quality factor associated therewith, which can be determined at least in part based a noise level or noise magnitude detected in each frequency band. When determining physiological metrics, such as oxygen saturation of tissue, the quality factor for each frequency band can be used to indicate a level of confidence in that particular frequency band.

Further types of filters can be included in place of FIR filter 350, or in addition to FIR filter 350, such as adaptive comb filtering (ACF), lattice filtering, empirical mode decomposition (EMD), ensemble averaging, filtering triggered based on an estimate of pulse rate or from a processed waveform, adaptive filters that make assumptions on the stability on one or more physiological parameters, Kalman filters, neural nets, fuzzy logic, genetic, or evolutionary algorithms or other filtering and signal processing techniques, such as pulse or signal qualification based on one or more properties such as waveform amplitude, morphology, regularity, stability, or higher-order moments, including combinations thereof measured over one or more time scales. In addition to time domain and frequency domain, other signal domains can be analyzed for correlation and noise reduction. The other signal domains can include wavelet domains, cepstral domains, and autocorrelation domains.

The adaptive filtering employed in FIG. 3 can perform well when the measured PPG and the capacitance signal are correlated in time, or noise components of both signals are correlated in time. To ensure better filtering of the measured PPG, a frequency-domain spectral subtraction element 344 is employed. This spectral subtraction can aid in filtering and noise reduction of the measured PPG when noise components are not well correlated in time to the reference signal, or to further clean the filtered PPG when the noise is correlated in time.

In element 344, a power spectrum or magnitude of a Fast Fourier Transform (FFT) of the reference signal is subtracted from that of a noisy signal of interest. Specifically, a FFT of signal 335 is subtracted from an FFT of signal 334 for overlapping time windows. Various scaling can be applied to signal 335 to adjust energy levels of noise components of signal 335 to that of signal 334. Once the FFTs of each signal are obtained, then the overlapping time windows for each transformed signal can be analyzed to identify power spectrums of each signal. Once the FFT version of signal 335 is subtracted from the FFT version of signal 334, then a reverse FFT is performed on the resultant signal to obtain final PPG signal 336. Phase relationships between the input signals and the reverse FFT signals can be maintained in element 344 using the overlapping time windows. In FIG. 3, final PPG 336 comprises a PPG that can be used to identify various physiological metrics for the patient, such as those described above for FIG. 2. An example of final PPG 336 is included in graph 403 of FIG. 4, namely final PPG 413.

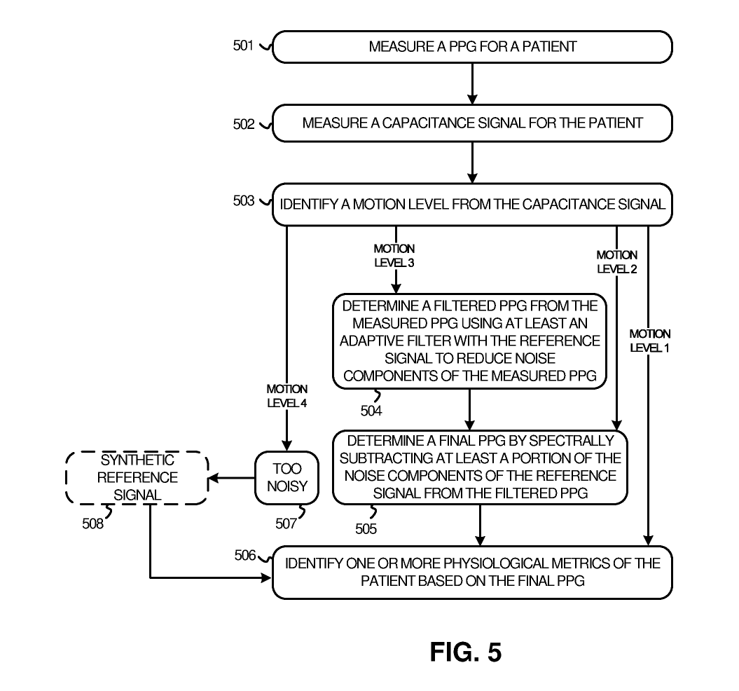

To further discuss the instantaneous power thresholds of element 342 of FIG. 3, FIG. 5 is presented. FIG. 5 is a flow diagram illustrating a method of operating a measurement system and associated filtering and signal processing elements, such as that found in FIG. 1 or FIG. 3. In FIG. 5, a motion level is identified based on a level of noise in a reference signal for a patient. This motion level is used to selectively apply a level of filtering or signal processing to a measured PPG signal. For example, a less processing-intensive filtering or signal processing technique can be applied to a measured PPG signal depending upon a level of noise expected in the PPG signal. This can save processing resources and reduce power consumption of any associated measurement apparatus or systems.

In FIG. 5, a PPG is measured for a patient (501). This PPG is measured by optical equipment, such as optical system 114 of FIG. 1 which employs optical elements 121-122 on tissue 130. Concurrent with the measurement of the PPG, a reference signal is measured (502). This reference signal can include a capacitance signal, as discussed in FIGS. 1 and 3, or can be an ambient light signal as discussed in FIGS. 8-9 below. Combinations of these reference signals can be used.

Based on the reference signal, a motion level is identified for the tissue or patient (503). This motion level can be identified based on an instantaneous measure of noise in the reference signal, such as an instantaneous power of a capacitance signal measured for the patient. This instantaneous power can be measured in a frequency domain, such by taking a FFT of the capacitance signal and identifying magnitudes of power for the various frequency content. In some examples, a predetermined frequency or frequencies can be used for identification of the instantaneous measure of noise, or a range of frequencies can be analyzed. Once the instantaneous measure of noise has been obtained, this measure is applied to one or more threshold levels. Four threshold levels are applied in FIG. 5, although it should be understood that a different number or combination can be employed.

For a first motion level, such as when the reference signal has an instantaneous power below a first threshold, a first amount of signal processing is performed. In the first motion level, a noise level in the reference signal indicates that the concurrently measured PPG does not have a high level of motion noise, and thus the measured PPG can be used to directly obtain physiological metrics in operation 506. A lower intensity of processing can be applied to the measured PPG during motion level 1, such as just bandpass filtering or gain adjustment.

For a second motion level, such as when the reference signal has an instantaneous power above the first threshold but below a second threshold, then a second amount of signal processing is performed. In FIG. 5, a spectral subtraction process is performed on the measured PPG using the reference signal as a subtraction signal, as indicated by operation 505. This spectral subtraction can include operations as discussed above for element 113 of FIG. 1 or 344 of FIG. 3. Once the spectral subtraction operation is performed, then a resultant PPG can be used to obtain physiological metrics in operation 506. Alternatively, only an adaptive filtering operation might be performed and a spectral subtraction operation might be omitted for the second motion level, such as discussed in operation 504.

For a third motion level, such as when the reference signal has an instantaneous power above the second threshold, but below a third threshold, then a third amount of signal processing is performed. In FIG. 5, an adaptive filtering process is performed on the measured PPG in operation 504. The measured PPG is adaptively filtered using the reference signal, such as discussed above for adaptive processor 112 in FIG. 1 or adaptive filter 343 in FIG. 3. After the measured PPG is adaptively filtered, then the filtered PPG is further processed by a spectral subtraction operation in operation 505. Once the spectral subtraction operation is performed, then a resultant PPG can be used to obtain physiological metrics in operation 506.

For a fourth motion level, such as when the reference signal has an instantaneous power above the third threshold, then the measured PPG might be too noisy for effective signal processing and filtering in operations 504 and 505. Operation 507 indicates that the measured PPG may be too noisy to be used to determine physiological parameters in operation 506. An indicator, warning, or other information can be presented to an operator of an associated measurement system that indicates the operator of the noisy condition or low quality of the measured PPG. For example, if motion of the patient or associated tissue is too severe, then any associated measurement using an optical system to obtain a PPG might also be too corrupted to provide for effective determination of any physiological parameters. Optical elements, such as optical emitters or detectors can be powered down during periods of high motion or high noise to save energy or extend lifetimes of the associated optical components. Once the reference signal indicates that motion has subsided below one of the predetermined thresholds, then signal processing of the PPG can resume to obtain filtered/processed PPG data or associated physiological metrics.

For the fourth motion level, processing of the measured PPG might still be desired. The reference signal indicates that motion noise is above a ceiling that might prevent accurate determination of physiological parameters or metrics. In operation 508, a synthetic reference signal can be applied. This synthetic reference signal can be generated by a measurement system, such as processing system 111 of FIG. 1 using various signal synthesis techniques.

The synthetic reference signal can comprise a signal based on a measurement of the PPG during periods of low noise, such as during the first motion level, or during periods of higher noise, but after a filtering/processing has obtained a cleaner PPG signal. Specifically, various frequency content of the “clean” PPG can be identified, such as fundamental frequencies of pulse signals for the patient, and a signal can be synthesized based on this fundamental frequency which is an approximation of a “real” PPG. The synthetic reference signal can be based on past pulse rates, past breathing rates, or heart rate parameters for the patient. The heart rate parameters can include systolic/diastolic information for the patient. The synthetic reference signal can be used to establish filtering or processing parameters for the measured PPG during periods of high motion noise so that a usable final PPG can be obtained. The synthetic reference signal can be updated or re-tuned during periods of low noise for the measured PPG to ensure an accurate representation is synthesized.

Furthermore, even when motion noise does not exceed the fourth threshold mentioned above, motion noise might spectrally overlap with pulsatile signals desired to be measured for the patient. The overlap can occur when a frequency of the motion of the patient substantially matches a pulse frequency of the measured PPG. This overlap can make filtering and processing of the measured PPG difficult, since the associated reference signal has energy at physiologically relevant frequencies of the measured PPG, such as a pulse of the patient. To aid in filtering or processing of the measured PPG when motion indicated by the reference signal spectrally overlaps, the synthetic reference signal can be employed.

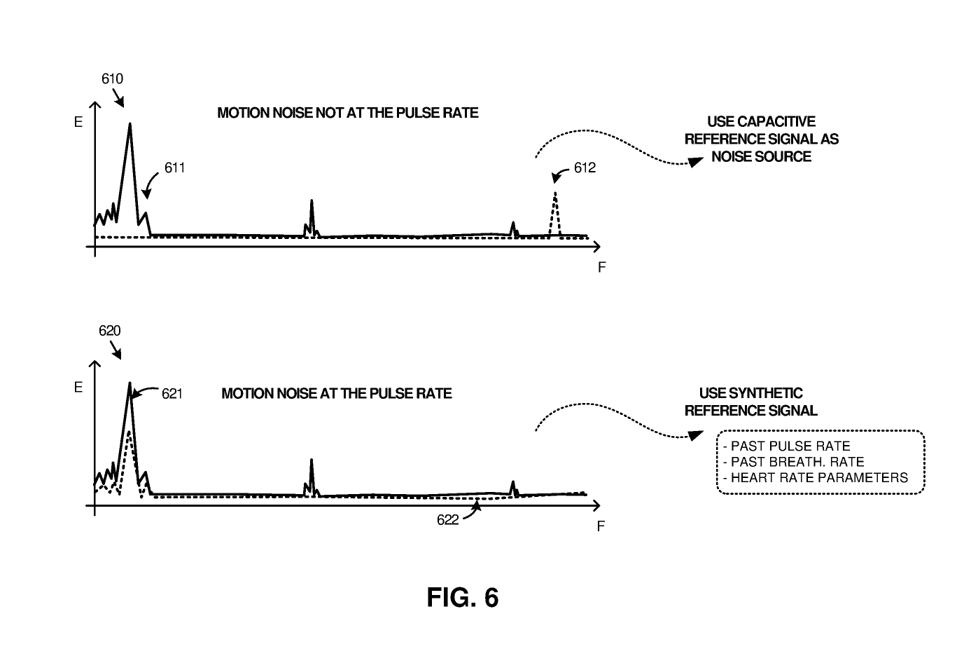

To further illustrate overlapping spectral content, FIG. 6 is provided. FIG. 6 shows example spectrums of PPG signals and capacitive reference signals. Graph 610 is an example where capacitive reference signal 612 does not have motion noise content at a fundamental frequency associated with a pulse rate of PPG 611. When this is the case, the capacitive reference signal can be used in adaptive filtering and spectral subtraction processes, such as those discussed above. Conversely, graph 620 is an example where capacitive reference signal 622 does have motion noise content at a fundamental frequency associated with a pulse rate of PPG 621. Since filtering or signal subtraction using capacitance signal 622 might eliminate portions of PPG 621 that are desired, a synthetic reference signal might instead be used, such as that discussed above which is synthesized based in part on past information for the patient. The synthetic reference signal can be based on past pulse rates, past breathing rates, or heart rate parameters established for the patient.

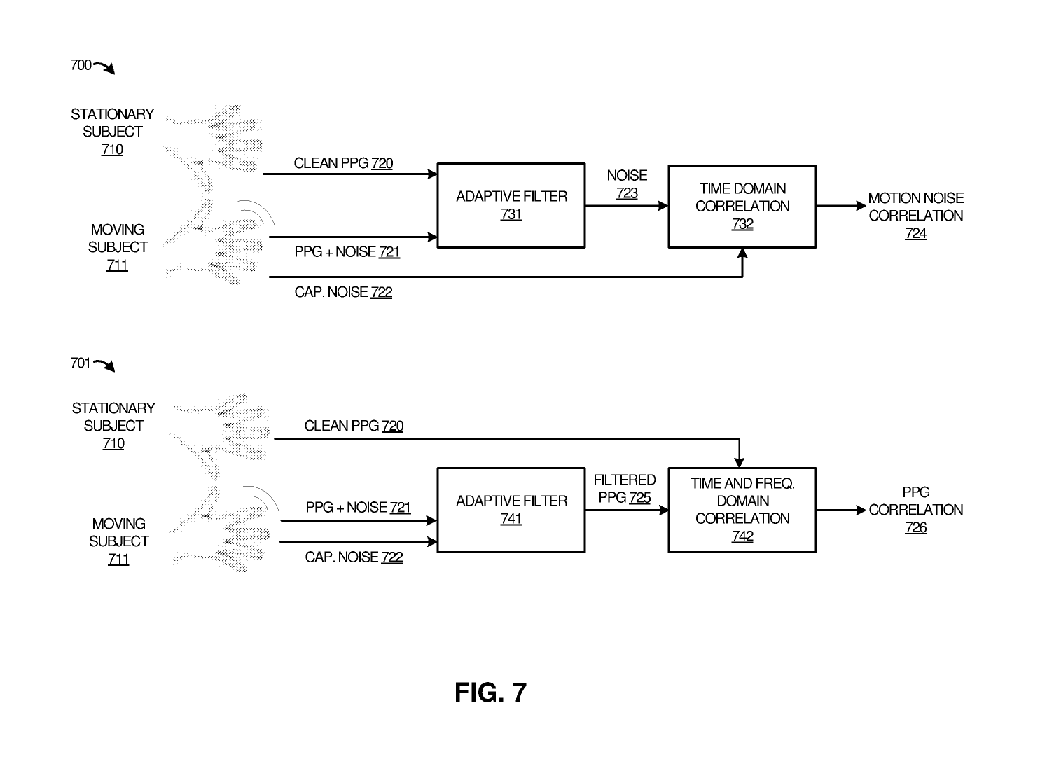

FIG. 7 is a system diagram illustrating physiological measurement systems which can be used to check correlations between measured PPG signals and reference signals. Both systems 700 and 701 can be employed to maximize effectiveness of the sensor design and filtering parameters used to reduce noise in PPG signals using a reference signal. Various correlation checking can be performed using a measured PPG signal and the reference signal to ensure correlation of the two signals. The correlation is relevant in the adaptive filtering techniques discussed above to filter a measured PPG using a reference signal. If the two signals are not correlated enough in time or frequency, then an adaptive filtering technique might be less effective, or the adaptive filtering technique might need to be modified accordingly. System 700 shows elements for checking correlation of motion noise in the time domain. System 701 shows elements for checking correlation of a filtered PPG to a “clean” PPG.

Turning first to system 700, two measurement subjects are shown, namely stationary subject 710 and moving subject 711. Stationary subject 710 and moving subject 711 can be tissue of a patient undergoing measurement, such as a non-moving hand and a moving hand of the patient. A “clean” PPG in this example refers to a PPG which does not have significant noise content from bulk motion of the tissue, such as due to tapping, stretching, flexing, and the like. Clean PPG 720 is introduced into adaptive filter 731 along with noisy PPG 721. In this example, a noisy signal (721) is filtered against a clean version (720) of that signal, which results in noise signal 723 as an output from adaptive filter 731. Adaptive filter 731 compares noisy PPG 721 against clean PPG 720 to filter out a pulse signal from noisy PPG 721 and obtain noise signal 723. This noise signal 723 indicates the motion noise in PPG 721. Then, capacitive signal 722 is input to a time domain correlation element 732 along with noise signal 723. These two signals are analyzed for an amount of time domain correlation in element 732, and the result is metric 724 that indicates the amount of time domain correlation.

If a sufficient amount of correlation is found for capacitive signal 722 and noisy PPG signal 721, then the adaptive processing can be considered to be an effective filtering technique for the associated input signals. Metric 724 indicates how well motion artifacts in PPG signal 721 are captured by a reference signal, such as using a capacitive sensor. Metric 724 also provides a quantitative value to aid the capacitive sensor design, such as to tune physical and electrical properties of the capacitive sensor to maximize correlation between motion noise components of a PPG signal to the capacitive signal.

Turning now to system 701 of FIG. 7, two measurement subjects are shown, namely stationary subject 710 and moving subject 711 discussed above. Noisy PPG signal 721 is introduced into adaptive filter 741 along with capacitive signal 722. In this example, a noisy signal (721) is filtered against capacitive signal 722 as a reference signal to obtain filtered PPG signal 725. Adaptive filter 741 can comprise elements discussed above for adaptive processor 112 of FIG. 1 and adaptive processor 343 of FIG. 3 to filter out noise found in capacitive signal 722 from noisy PPG 721. Next, filtered PPG signal 725 and clean PPG signal 720 are compared in both time domain and frequency domain by element 742 to determine a correlation between the two signals.

Element 742 obtains metric 726 that indicates how well motion noise in noisy PPG signal 721 was canceled using capacitive reference signal 722. This operation is the reverse operation of motion correlation discussed for system 701 above. Metric 726 can be employed to adjust parameters of adaptive filter 741 to maximize cancellation of noise in PPG measurements by a capacitive signal. System 701 can be used to adjust filtering parameters of adaptive processor 112 of FIG. 1 and adaptive processor 343 of FIG. 3, or to select among a plurality of filtering techniques, filter circuitry, or filter parameters, to maximize effectiveness of the filtering discussed herein.

As mentioned above, a reference signal can comprise a capacitive signal measured using one or more capacitor plates, or the reference signal can comprise an ambient light signal. Turning now to an ambient light signal example, FIG. 8 is presented. FIG. 8 is a system diagram illustrating physiological measurement system 800. The elements of FIG. 8 can comprise similar elements as found in FIG. 1, and these elements are included in FIG. 8 for clarity. It should be understood that other elements different than those of FIG. 1 can instead be included.

System 800 includes measurement system 810, sensor elements 820, and tissue 130. Measurement system 810 includes processing system 111 and optical system 114. Processing system 111 and optical system 114 communicate over link 116. In this example, processing system 111 includes adaptive processor 112, spectral processor 113, and ambient light processor 814. Sensor elements 820 include optical emitter 121 and optical detector 122 which are not accompanied by capacitive sensing elements in FIG. 8. In operation, sensor elements 820 are configured to monitor various properties of tissue 130 and provide signals indicating these properties to measurement system 810 for processing and analysis.

Optical system 114 measures various properties of tissue 130 using optical emitter 121 and optical detector 122. In some examples, optical system 114, along with optical emitter 121 and optical detector 122, comprise a pulse oximeter and can identify a measured PPG for tissue 130. This PPG can be used to determine various properties of tissue 130 or the patient associated with tissue 130, such as changes in blood volume of tissue 130 which correspond to various parameters such as pulse rate, respiration rate, and oxygen saturation, among other parameters.

However, as discussed herein, motion of tissue 130 as well as other bulk motion of the patient can introduce noise and signal corruption into measurements done by optical system 114. In FIG. 8, an ambient light signal is employed as a reference signal, in contrast to the capacitive signals mentioned in FIG. 1. Ambient light 880 is measured by optical detector 122, and an ambient light signal is identified by ambient light processor 814. Although FIG. 8 shows ambient light processor 814 in processing system 111, it could instead be included in optical system 114 or other elements of FIG. 8.

During measurement of tissue 130 to obtain a measured PPG, optical detector 122 detects optical signals emitted by optical emitter 121 after propagation through tissue 130. Optical system 114 receives signals over link 142 from optical detector 122 representative of the optical signals after propagation through tissue 130. To obtain the ambient light signal, optical detector 122 measures ambient light 880 and provides a measure of ambient light over link 142 to optical system 114.

In some examples, optical emitter 121 is turned off when ambient light 880 is desired to be measured, which is referred to as the “dark” timeframes for optical emitter 121. During the dark timeframes, the only light sources are from nearby lamps, overhead lights, and the like, because optical emitter 121 is not emitting light. Most of the light from ambient sources would arrive at optical detector 122 without propagating through tissue 130, so only a tiny PPG signal is observed when optical emitter 121 is not emitting light. However, amplitude oscillations caused by changes in ambient light 880 coupling to optical detector 122 during motion of tissue 130 is many orders of magnitude larger than this tiny PPG signal. Since the ambient light signal has a tiny associated PPG, but large motion contributions, the ambient light signal can be used as a reference signal for motion noise reduction in a PPG measured when optical emitter 121 is emitting light.

A low pass filter can be applied to optical signals measured by optical detector 122 to obtain the ambient light signal. Overhead lights in many medical facilities do oscillate due to corresponding oscillations of the main AC power to the overhead lights, or due to oscillations of the light driving circuitry of the overhead lights, such as in fluorescent or LED lighting elements of a hospital room. These oscillations of ambient light sources are typically about 60 Hz or greater in frequency, and can be filtered out from tissue motion signals which typically are not higher in frequency than approximately 10-12 Hz.

In some cases, an ambient light signal can be affected by other motion or objects than the motion of the patient. For example, ambient light 880 can be blocked or reduced by external objects nearby the patient, such as blankets or personnel leaning over optical detector 122. In these cases, capacitive sensor elements can be included, such as in FIG. 1, which can monitor movement of the patient in parallel with the ambient light signals. If one signal source exhibits poor quality, such as when the capacitance signal is corrupted due to too much noise or due to touching of the capacitive sensor by medical personnel, then the ambient light signal can be used by processing system 111 to filter/process a measured PPG. Likewise, if the ambient light signal exhibits poor quality, such as when ambient light 880 is blocked by personnel or objects nearby the patient, then the capacitive signal can be used by processing system to filter/process a measured PPG.

Once the ambient light signal is obtained for use as a reference signal, then processing system 111 can employ the ambient light signal in adaptive processor 112 and spectral processor 113. Adaptive processor 112 includes one or more adaptive filtering elements which can process ambient light signals monitored by optical system 114 to reduce noise levels in a PPG measured by optical system 114. Spectral processor 113 includes one or more signal processing elements which can perform additive and subtractive signal operations in a time domain or a frequency domain. Further discussion of the adaptive processing and spectral subtraction elements are discussed in the various examples herein.

FIG. 9 is presented to further illustrate the ambient light examples of FIG. 8. FIG. 9 illustrates various measured signals for a patient. Specifically, graph 900 indicates a frequency spectrum for optically measured signals for tissue 130. Motion noise in the ambient light signal might have associated noise components 912 around the 10 Hz frequency, while a pulse of the patient might have an associated fundamental frequency around the 2 Hz frequency. These signal and noise components can be separated from associated ambient light oscillations using a low-pass filter. Graph 900 indicates these ambient light oscillations at the 120 Hz frequency.

Graph 901 indicates a time domain graph resultant from the low-pass filter applied to the ambient light signals shown in graph 900. A first time portion 921 of graph 901 illustrates a motion-free timeframe, while a second time portion of 922 of graph 901 illustrates a motion timeframe. As can be seen in graph 901, motion of the patient is distinct in signal content from a time when motion is not occurring for the patient. Graph 901 omits the pulse signals of the patient to more clearly illustrate the change in ambient light signals during motion of the patient.

Graph 902 is another frequency domain graph, illustrating a frequency spectrum of measured PPG signal 941 and ambient light signal 942. Both measured PPG signal 941 and ambient light signal 942 include motion detected for a patient, while only measured PPG signal 941 has significant energy at a pulse of the patient. As graph 902 shows, a pulse signal of the patient has fundamental frequency 931 of 2 Hz, with at least one harmonic 932 higher in frequency. Noise frequency content related to motion of the patient is observed with a frequency of about 5 Hz. This motion noise in ambient light signal 942 correlates well in the frequency domain to motion noise in measured PPG signal 941, and both signals have high relative energy at the motion noise frequency. Thus, ambient light signal 942 can be used in the filtering and signal processing techniques discussed herein as a reference signal instead of, or in addition to a capacitive signal.

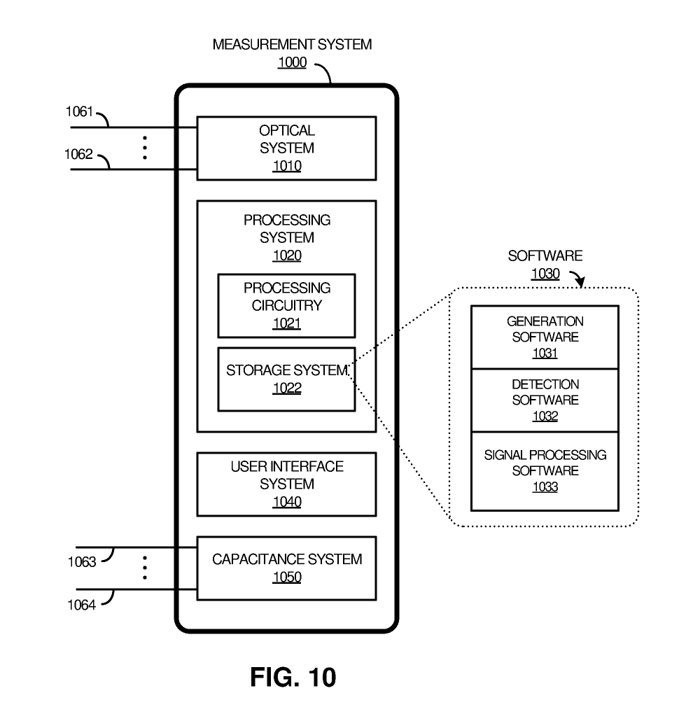

FIG. 10 is a block diagram illustrating measurement system 1000, as an example of elements of measurement system 110 in FIG. 1 or signal processor 300 in FIG. 3, although these can use other configurations. Measurement system 1000 includes optical system 1010, processing system 1020, software 1030, user interface 1040, and capacitance system 1050. Processing system 1020 further includes processing circuitry 1021 and storage system 1022. In operation, processing circuitry 1021 is operatively linked to optical system 1010, user interface 1040, and capacitance system 1050 by one or more communication interfaces, which can comprise a bus, discrete connections, network links, software interfaces, or other circuitry. Measurement system 1000 can be distributed or consolidated among equipment or circuitry that together forms the elements of measurement system 1000. Measurement system 1000 can optionally include additional devices, features, or functionality not discussed here for purposes of brevity.

Optical system 1010 comprises a communication interface for communicating with other circuitry and equipment, such as with optical system 104 of FIG. 1. Optical system 1010 can include transceiver equipment exchanging communications over one or more of the associated links 1061-1062. It should be understood that optical system 1010 can include multiple interfaces, pins, transceivers, or other elements for communicating with multiple external devices. Optical system 1010 also receives command and control information and instructions from processing system 1020 or user interface 1040 for controlling the operations of optical system 1010. Links 1061-1062 can each use various protocols or communication formats as described herein for links 116 or 141-142 of FIG. 1, including combinations, variations, or improvements thereof. In some examples, optical system 1010 includes optical interface equipment, such as that discussed above for optical system 114.

Processing system 1020 includes processing circuitry 1021 and storage system 1022. Processing circuitry 1021 retrieves and executes software 1030 from storage system 1022. In some examples, processing circuitry 1021 is located within the same equipment in which optical system 1010, user interface 1040, or capacitance system 1050 are located. In further examples, processing circuitry 1021 comprises specialized circuitry, and software 1030 or storage system 1022 can be included in the specialized circuitry to operate processing circuitry 1021 as described herein. Storage system 1022 can include a non-transitory computer-readable medium such as a disk, tape, integrated circuit, server, flash memory, or some other memory device, and also may be distributed among multiple memory devices.

Software 1030 may include an operating system, logs, utilities, drivers, networking software, tables, databases, data structures, and other software typically loaded onto a computer system. Software 1030 can contain application programs, server software, firmware, processing algorithms, or some other form of computer-readable processing instructions. When executed by processing circuitry 1021, software 1030 directs processing circuitry 1021 to operate as described herein, such as instruct optical or capacitance systems to generate optical or electrical signals for measurement of physiological parameters of patients, receive signals representative of optical or capacitance measurements of patients, and process at least the received signals to determine physiological parameters of patients, among other operations.

In this example, software 1030 includes generation module 1031, detection module 1032, and signal processing module 1033. It should be understood that a different configuration can be employed, and individual modules of software 1030 can be included in different equipment in measurement system 1000. Generation module 1031 determines parameters for optical or capacitance signals, such as modulation parameters, signal strengths, amplitude parameters, voltage parameters, on/off conditions, or other parameters used in controlling the operation of optical systems and capacitance systems over ones of links 1061-1064. Generation module 1031 directs optical system 1010 and capacitance system 1050 to perform physiological measurements, and can selectively drive various detection sensors, emitters, capacitors, and other sensor elements. Detection module 1032 receives data or signals representative of optical and capacitive measurements. Signal processing module 1033 processes the received characteristics of optical and reference signals to determine physiological parameters, adaptively filter optical data based on reference signals, spectrally subtract optical and reference signals from each other, and otherwise reduce motion noise in optical measurements using capacitance measurements or ambient light measurements, among other operations.

User interface 1040 includes equipment and circuitry to communicate information to a user of measurement system 1000, such as alerts, measurement results, and measurement status. Examples of the equipment to communicate information to the user can include displays, indicator lights, lamps, light-emitting diodes, haptic feedback devices, audible signal transducers, speakers, buzzers, alarms, vibration devices, or other indicator equipment, including combinations thereof. The information can include blood parameter information, waveforms, summarized blood parameter information, graphs, charts, processing status, signal quality information, motion noise level information, or other information. User interface 1040 also includes equipment and circuitry for receiving user input and control, such as for beginning, halting, or changing a measurement process or a calibration process. Examples of the equipment and circuitry for receiving user input and control include push buttons, touch screens, selection knobs, dials, switches, actuators, keys, keyboards, pointer devices, microphones, transducers, potentiometers, non-contact sensing circuitry, or other human-interface equipment.

Capacitance system 1050 comprises a communication interface for communicating with other circuitry and equipment, such as with capacitance system 115 of FIG. 1. Capacitance system 1050 can include transceiver equipment exchanging communications over one or more of the associated links 1063-1064. It should be understood that capacitance system 1050 can include multiple interfaces, pins, transceivers, or other elements for communicating with multiple external devices. Capacitance system 1050 also receives command and control information and instructions from processing system 1050 or user interface 1040 for controlling the operations of capacitance system 1050. Links 1063-1064 can each use various protocols or communication formats as described herein for link 117 or links 143-144 of FIG. 1, including combinations, variations, or improvements thereof. In some examples, capacitance system 1010 includes capacitance interface equipment, such as that discussed above for capacitance system 115.

The included descriptions and drawings depict specific embodiments to teach those skilled in the art how to make and use the best mode. For the purpose of teaching inventive principles, some conventional aspects have been simplified or omitted. Those skilled in the art will appreciate variations from these embodiments that fall within the scope of the invention. Those skilled in the art will also appreciate that the features described above can be combined in various ways to form multiple embodiments. As a result, the invention is not limited to the specific embodiments described above.

What is claimed is:

1. A physiological measurement system, comprising:
an optical system configured to obtain a measured photoplethysmogram (PPG) for a patient;
a reference system configured to obtain a reference signal for the patient comprising at least one of a capacitive signal and an ambient light signal measured concurrent with the measured PPG, the reference signal including noise components related to at least motion of the patient;
a signal processing system configured to determine a filtered PPG from the measured PPG using at least an adaptive filter with the reference signal to reduce noise components of the measured PPG;
the signal processing system configured to determine a final PPG by spectrally subtracting at least a portion of the noise components of the reference signal from the filtered PPG; and
the signal processing system configured to identify one or more physiological metrics of the patient based on the final PPG.

2. The physiological measurement system of claim 1, wherein the capacitive signal is obtained by monitoring one or more generally ring-shaped capacitance plates proximate to tissue of the patient.

3. The physiological measurement system of claim 1, wherein the ambient light signal is obtained from optical signals used to obtain the measured PPG for the patient.

4. The physiological measurement system of claim 1, comprising:
the signal processing system configured to correlate frequency-domain noise components of the reference signal to frequency-domain noise components of the filtered PPG data and spectrally subtract a scaled version of the frequency-domain noise components of the reference signal from the filtered PPG to determine the final PPG.

5. The physiological measurement system of claim 1, wherein the adaptive filter comprises a finite impulse response (FIR) filter; and comprising:
the FIR filter configured to process the reference signal against an error signal based on the filtered PPG to identify a cancellation signal, and sum the cancellation signal with the measured PPG to identify the filtered PPG.

6. The physiological measurement system of claim 1, wherein the adaptive filter comprises a bank of filters, with each of the filters corresponding to a different frequency band which has an associated quality factor associated therewith, the associated quality factor determined at least in part based on a magnitude of noise detected in each frequency band.

7. The physiological measurement system of claim 1, comprising:
the signal processing system configured to establish a synthetic reference signal that simulates a noise-free version of the measured PPG; and
the signal processing system configured to identify when at least a frequency of the motion of the patient substantially matches a frequency of the measured PPG, and responsively obtain the filtered PPG using the adaptive filter to reduce noise components in the measured PPG based at least on the reference signal and the synthetic reference signal.

8. The physiological measurement system of claim 1, comprising:
the signal processing system configured to process the measured PPG with a statistical independent component analysis (ICA) to extract statistically independent signals from each of the measured PPG and reference signal, a first of the statistically independent signals comprising motion noise of the patient and a second of the statistically independent signals comprising the filtered PPG.

9. The physiological measurement system of claim 1, comprising:
the signal processing system configured to identify a magnitude of the motion of the patient based on the reference signal;
the signal processing system configured to selectively apply ones of the adaptive filter and the spectral subtraction to obtain the final PPG based on a value of the magnitude of the motion of the patient;
when the magnitude of the motion of the patient exceeds a maximum threshold, the signal processing system configured to indicate to an operator of the physiological measurement system that the final PPG cannot be obtained due to excessive motion of the patient.

10. A method of operating a physiological measurement system, the method comprising:
obtaining a measured photoplethysmogram (PPG) for a patient;
obtaining a reference signal for the patient comprising at least one of a capacitive signal and an ambient light signal measured concurrent with the measured PPG, the reference signal including noise components related to at least motion of the patient;
determining a filtered PPG from the measured PPG using at least an adaptive filter with the reference signal to reduce noise components of the measured PPG;
determining a final PPG by spectrally subtracting at least a portion of the noise components of the reference signal from the filtered PPG; and
identifying one or more physiological metrics of the patient based on the final PPG.

11. The method of claim 10, wherein the capacitive signal is obtained by monitoring one or more generally ring-shaped capacitance plates proximate to tissue of the patient.

12. The method of claim 10, wherein the ambient light signal is obtained from optical signals used to obtain the measured PPG for the patient.

13. The method of claim 10, wherein spectrally subtracting comprises correlating frequency-domain noise components of the reference signal to frequency-domain noise components of the filtered PPG data and spectrally subtracting a scaled version of the frequency-domain noise components of the reference signal from the filtered PPG to determine the final PPG.

14. The method of claim 10, wherein the adaptive filter comprises a finite impulse response (FIR) filter that processes the reference signal against an error signal based on the filtered PPG to identify a cancellation signal, and wherein the cancellation signal is summed with the measured PPG to identify the filtered PPG.

15. The method of claim 10, wherein the adaptive filter comprises a bank of filters, with each of the filters corresponding to a different frequency band which has an associated quality factor associated therewith, the associated quality factor determined at least in part based on a magnitude of noise detected in each frequency band.

16. The method of claim 10, further comprising:
establishing a synthetic reference signal that simulates a noise-free version of the measured PPG; and
identifying when at least a frequency of the motion of the patient substantially matches a frequency of the measured PPG, and responsively obtaining the filtered PPG using the adaptive filter to reduce noise components in the measured PPG based at least on the reference signal and the synthetic reference signal.

17. The method of claim 10, further comprising:
processing the measured PPG with a statistical independent component analysis (ICA) to extract statistically independent signals from each of the measured PPG and reference signal, a first of the statistically independent signals comprising motion noise of the patient and a second of the statistically independent signals comprising the filtered PPG.

18. The method of claim 10, further comprising:
identifying a magnitude of the motion of the patient based on the reference signal;
selectively applying ones of the adaptive filter and the spectral subtraction to obtain the final PPG based on a value of the magnitude of the motion of the patient;
when the magnitude of the motion of the patient exceeds a maximum threshold, indicating to an operator that the final PPG cannot be obtained due to excessive motion of the patient.

19. An apparatus comprising:
one or more computer readable storage media; and
program instructions stored on the one or more computer readable storage media for at least identifying one or more physiological metrics of a patient, that when executed by a processing system, direct the processing system to at least:
obtain a measured photoplethysmogram (PPG) for a patient;
obtain a capacitance signal for the patient measured concurrent with the measured PPG using one or more capacitive plates proximate to tissue of the patient, the capacitance signal including noise components related to at least motion of the patient;
determine a filtered PPG from the measured PPG using at least an adaptive filter with the capacitance signal to reduce noise components of the measured PPG;
determine a final PPG by spectrally subtracting at least a portion of the noise components of the capacitance signal from the filtered PPG; and
identify the one or more physiological metrics of the patient based on the final PPG.

20. The apparatus of claim 19, wherein the processing instructions further direct the processing system to:
identify a magnitude of the motion of the patient based on the capacitance signal;
selectively apply ones of the adaptive filter and the spectral subtraction to obtain the final PPG based on a value of the magnitude of the motion of the patient;
when the magnitude of the motion of the patient exceeds a maximum threshold, indicate to an operator that the final PPG cannot be obtained due to excessive motion of the patient.
